(19) United States
(12) Plant Patent Application Publication
(10) Pub. No.: US 2003/0172419 P1
(43) Pub. Date: Sep. 11, 2003

(54) P.F. 28-007 peach tree origin of variety

(76) Inventor: Paul Jan Friday, Coloma, MI (US)

Correspondence Address: Paul Jan Friday, P.O. Box 850, Coloma, MI, 49038, US

(21) Appl. No.: 10/087,960

(22) Filed: Mar. 5, 2002

(51) Int. Cl. A01H 5/00

(52) U.S. Cl. PLT/198

(57) ABSTRACT

A new and distinct variety of peach, Prunus persica, tree having the following unique combination of desirable features:

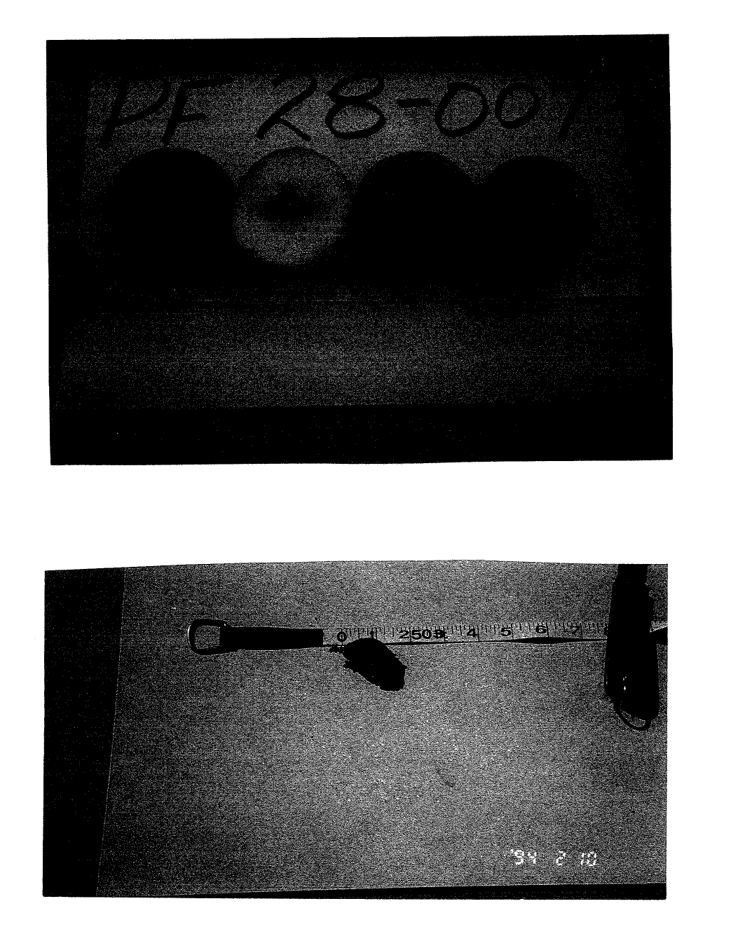

1. The new and distinct variety of peach tree is of average height and of upright growth and a regular and productive bearer of peaches.

2. Producing a very firm fruit having a resilient flesh texture.

3. Blossoms are non-showy when in full bloom.

4. A substantially oval spherical fruit with skin of dark red cover overlying a yellow which covers approximately twenty percent (20%) of its surface at maturity.

5. When harvested, the peduncle often remains affixed to the pit.

6. Late maturing fruit of good taste.

7. A late maturing fruit of good storage and shelf life.

### ORIGIN OF VARIETY

The new peach tree (hereinafter referred to as the P.F. 28-007 peach tree) was originated by Paul Friday in the experimental orchard, which is maintained for the purposes of breeding peach trees, at Paul Friday Farms Inc., located in Coloma, Mich. Coloma is located in the southwest section of Michigan.

In an ongoing mass selection breeding program, superior seedlings of unrecorded parentage are maintained as seed sources for the production of seeds which are collected and planted in mass. The seed producing parent trees are maintained solely as proprietary trees for breeding purposes and have not been released from the experimental orchard, where such trees can be evaluated for their adaptability to local and regional growing conditions. Seeds resulting from open pollination of the trees in the experimental orchard are regularly planted in mass to produce new populations of seedlings which are cultured and monitored to maturity. Trees with superior attributes are retained for further observation and testing, and contribute seeds to advancing generations of new populations of seedlings.

The tree of this application, P.F. 28-007, was a selection from one such a seedling population, and was based on the numerous superior genetic attributes of this tree which are described in the botanical description to follow. While not comprehensive, the details of the botanical description to follow are believed to be a reasonably complete botanical description of the tree of this disclosure.

### ASEXUAL REPRODUCTION OF THE VARIETY

The new and distinct variety of peach tree was asexually propagated by budding as performed in the experimental orchard of Paul Friday Farms Inc., located in Coloma, Mich. The asexual propagation demonstrates that such reproduction of the characteristics of the tree are consistent and are established and transmitted through succeeding propagation.

### SUMMARY OF THE VARIETY

The new and distinct variety of peach tree is of average height and of upright growth and a regular and productive bearer of peaches. A distinct characteristic of the P.F 28-007 peach tree is its medium vigor having growth of about twenty-four inches (24″) per year. The blossoms bloom in mid-season and are characterized by being contracted or partially spread to approximately ¾-inch when in full bloom. At the same time the five petals of the blossoms are of lesser length than the length of petals of the normal showy blossom as exemplified by the Loring peach blossom.

The blossoms of the present peach tree at full bloom may be characterized as being non-showy. More specifically, the blossoms of the present peach tree have radially projecting and angularly spaced five blossom petals to form a blossom having a diameter of about ¾″ measured across the blossoms.

The flesh of the fruit of the present peach tree is firm and is yellow. This variety is very freestone.

The skin is smooth having moderate to little down and is of dark red color overlying a yellow ground color. The yellow background covers approximately twenty percent (20%) of its surface at maturity. At maturity, the peach is spherical having an average diameter of about 2¾″.

When harvested, the fruit is broken loose from the tree with the breaking point above the peduncle rather than below about fifty percent (50%) of the time.

The fruit produced by this tree has firm, and non-melting flesh, and thereby has the attendant resistance to blemishes and soft spots in harvesting, shipping and handling due to bruising. The firmness of the fruit flesh is sufficient to allow the flesh to yield and be restored when bumped or dropped without the resulting soft spots as would be experienced in most late season peaches of this market class. Thus, fruit of tree remains more attractive to the ultimate buyer, the consumer, and thereby will command premium process for the late fresh desert market.

The fruit matures in the latter part of the peach growing season of southwestern Michigan. The fruit as mentioned heretofore is of dark red color overlying a yellow, which covers approximately twenty percent (20%), of its surface and has a very attractive appearance.

### BRIEF DESCRIPTION OF THE PHOTOGRAPHS

In the accompanying photographic illustrations, the top photograph shows three whole fruit depicting the substantially solid red blush of fruit skin coloration. The recessed pistil point of the fruit, the medium suture, and the exceptional shape and characteristic symmetry of the fruit of this tree are depicted in this photograph. A single peach as been bisected across the axis of the fruit to show the freestone characteristic and clear yellow flesh. A tape measure has been placed showing the extraordinary size that this new variety can obtain.

The bottom photograph shows the peduncle strongly affixed to the pit.

### DESCRIPTION OF VARIETY

The detailed botanical description of the foliage and fruit of the new variety of peach tree is based upon observations of the specimens grown at Coloma, Mich. with the color terminology, other than the terminology expressed in common terms, in accordance with the Panatone Matching System (PMS) as used internationally to identify printed colors.

Tree:

Height.—Unpruned 14′.

Width.—Unpruned 12′.

Size.—Medium.

Vigor.—Medium.

Density.—Heavy — requires substantial pruning.

Form.—Upright.

Production.—Productive — requires a lot of thinning, even though it grows very large fruit.

Bearer.—Consistent.

Disease resistance to bacterial leaf and fruit spot.—Very good.

Trunk:

Bark.—Medium.

Size.—Medium.

Surface.—Particularly smooth.

Branches:

Size.—Medium.

Surface.—Particularly smooth.

Lenticels.—Length ½″ — less pronounced than usual.

Color.—Silver Grey.

Leaves:

Size.—Medium — average length 7″ — average width 1 17/16″.

Form.—Lanceolate — pointed.

Thickness.—Medium.

Texture.—Medium.

Margin.—Serrate.

Petiole.—Medium length — medium thickness.

Gland.—Oval — 1 to 3 on each side of leaf base or upper portion of petiole.

Gland color.—Yellow — red in fall.

Color.—Upper surface green (554) — lower surface dull green (369).

Flower buds:

Size.—Medium.

Length.—Medium.

Flowers:

Blooming period.—May 3, 2001 to May 6, 2001.

Size.—¾″ diameter.

Pollen.—Present, self-fertilizing.

Color.—Pink.

Fruit:

Maturity when described.—Firm ripe.

Date of first picking.—Sep. 12, 2001.

Date of last picking.—Sep. 20, 2001.

Size.—Very large — average diameter 2¾″.

Form.—Spherical.

Suture.—Relatively smooth.

Skin:

Thickness.—Medium.

Texture.—Medium.

Tendency to crack.—None.

Down.—Moderate.

Color.—Medium red (214 to 207) mottled to yellow ground color (129).

Flesh:

Texture.—Firm, non-melting.

Ripens.—Relatively even.

Flavor.—Excellent.

Aroma.—Pleasant.

Eating quality.—Very good.

Color.—Clear yellow (120) — red around the pit and slight red into flesh (187).

What is claimed is:

1. A new and distinct variety of peach tree substantially as illustrated and described as medium size, medium vigor and upright in growth—a regular and productive bearer of very large size yellow flesh freestone fruit with good flavor and shipping quality and having a late season maturity date of about Sep. 12, 2001.
